(19) United States
(12) Patent Application Publication
(10) Pub. No.: US 2009/0151023 A1
(43) Pub. Date: Jun. 11, 2009

(54) Transformation system for Camelina sativa

(76) Inventors: Viktor Kuvshinov, Vantaa (FI); Anne Kanerva, Itasalmi (FI); Kimmo Koivu, Itasalmi (FI); Svetlana Kuvshinova, Vantaa (FI); Eija Pehu, Helsinki (FI)

Correspondence Address: DODDS & ASSOCIATES, 1707 N STREET NW, WASHINGTON, DC, 20036, US

(21) Appl. No.: 12/290,379

(22) Filed: Oct. 30, 2008

Related U.S. Application Data
(63) Continuation-in-part of application No. 10/416,091, filed on Sep. 8, 2003.

(30) Foreign Application Priority Data
Nov. 13, 2000 (FI) FI110009

(51) Int. Cl. C12N 15/82; A01H 5/00

(52) U.S. Cl. 800/294; 800/306

(57) ABSTRACT

The present invention relates to plant biotechnology and specifically to a method for genetically transforming Camelina sativa with Agrobacterium-mediated transformation system. It comprises Camelina sativa for producing homologous and heterologous recombinant products including oil and protein products and assessing and screening the efficacy of plant transformation. Also disclosed are transgenic Camelina sativa plants, seeds as well as cells, cell-lines and tissue of Camelina sativa.

This application is a Continuation-in Part application, of U.S. application Ser. No. 10/416,091.

This patent application contains at least one drawing executed in color. Copies of this patent or patent application publication with color drawing(s) will be provided by the Office upon request and payment of the necessary fee.

### TECHNICAL FIELD OF THE INVENTION

The present invention is related to plant biotechnology and plant cell transformation. More particularly the invention relates to a method for genetically transforming Camelina sativa by Agrobacterium mediated transformation of plant tissue and subsequent method to regenerate transformed cells into whole transgenic plants. Moreover, the invention relates to a method to transform Camelina plant tissue without a selection marker and regeneration of selection marker free transgenic Camelina plants.

### BACKGROUND OF THE INVENTION

Genetic transformation of plants allows introduction of genes of any origin into the target species providing novel products for various applications including agricultural, horticultural, nutritional, pharmaceutical and chemical applications. Furthermore, transgenic plants may be used to study basic plant biology, gene function, and regulation. In many plant species, traditional plant breeding is limited due to the fact that the existing gene pool is narrow and prevents further development. Alteration of single characteristics can be time-consuming and even impossible without changing any other properties. Major applications of plant genetic transformation have focused on improvement of agricultural characteristics, such as disease resistance, insect resistance, and herbicide tolerance. Another widely studied area is modification of plant quality characteristics, such as modification of oil and protein compositions as well as improving stress tolerance and modifying growth characteristics. Yet another application is use of transgenic plants as bioreactors for producing foreign proteins, modified oils or plant secondary metabolites.

Several vector systems have been developed to be used in higher plants for transferring genes into plant tissue. The most widely used method is Agrobacterium tumefaciens or Agrobacterium rhizogenes mediated systems. Several Agrobacterium-mediated systems and methods for transforming plants and plant cells have been disclosed for example in WO 84/02920, EP 289478, U.S. Pat. No. 5,352,605, U.S. Pat. No. 5,378,619, U.S. Pat. No. 5,416,011, U.S. Pat. No. 5,569,834, U.S. Pat. No. 5,959,179, U.S. Pat. No. 6,018,100, and WO 00/42207. Several transformation strategies have been developed for Agrobacterium-mediated transformation system. The binary vector strategy is based on a two-plasmid system where T-DNA is in a different plasmid from the rest of the Ti plasmid. In the cointegration strategy a small portion of the T-DNA is placed in the same vector as the foreign gene, which vector subsequently recombines with the Ti plasmid.

The production of transgenic plants has become routine for many plant species, but no universal transformation method for different plant species exists, since transformation and regeneration capacity varies among species and even with different explants. Moreover, there may be a method for in vitro regeneration of a plant species, but the method does not necessarily work with transgenic plants. Therefore, there is a need for developing alternative transformation systems, along with methods to regenerate the transgenic plants. U.S. Pat. No. 5,188,958, U.S. Pat. No. 5,463,174 and U.S. Pat. No. 5,750,871 disclose transformation of Brassica species by Agrobacterium-mediated transformation system. These systems however, even if applicable to Brassica-species, do not work for Camelina sativa plants.

Selection markers are widely used in Agrobacterium mediated plant transformation to obtain efficient transformation rates. The most common selection markers are antibiotic resistance and herbicide resistance genes. However, there is a growing public concern of the selection marker genes, and accordingly, there is a growing area of research to find methods to either remove the selection marker from the transgenic plant after transformation or to find methods where no selection marker is needed. Recently a method to transform apple plants without selection marker has been disclosed in U.S. patent application Ser. No. 11/973,539.

Camelina sativa (L. Crantz) belongs to the family Brassicaceae in the tribe Sisymbrieae and both spring- and winter forms are in production. It is a low-input crop adapted to low fertility soils. Results from long-term experiments in Central Europe have shown that the seed yields of Camelina sativa are comparable to the yields of oil seed rape.

Due to the high oil content of Camelina sativa seeds (varying between 30-40%), there has been a renewed interest in Camelina sativa oil. Camelina sativa seeds have high content of polyunsaturated fatty acids, about 50-60% with an excellent balance of useful fatty acids including 30-40% of alpha-linolenic acid, which is an omega-3 oil. Omega-3 oils from plants metabolically resemble marine omega-3oils and are rarely found in other seed crops. Furthermore, Camelina sativa seeds contain high amount of tocopherols (appr. 600 ppm) with a unique oxidative stability. Moreover, the oil and meal are low in glucosinolates (Matthaus and Zubr, Industrial Crops and Products 12:9-18, 2000).

As Camelina sativa is a minor crop species, very little has been done in terms of its breeding aside from testing different accessions for agronomic traits and oil profiles. Mutation breeding induced variation in the fatty acid content by three- to four-fold (Buchsenschutz-Northdurft et al., 3rd European Symposium on Industrial Crops and Products, France, 1996). Application of tissue culture techniques to Camelina sativa are limited to two approaches: Camelina sativa has been used in a somatic fusion with other Brassica species (Narasimhulu et al., Plant Cell Rep. 13:657-660, 1994; Hansen, Crucifer. News 19:55-56, 1997; Sigareva and Earle, Theor. Appl. Genet. 98:164-170, 1999) and regenerated interspecific hybrid plants have been obtained (Sigareva and Earle, Theor. Appl. Genet. 98:164-170, 1999). Recently, Camelina sativa shoots have been regenerated from leaf explants (Tattersall and Millam, Plant Cell Tissue and Organ Culture 55:147-149, 1999). Even if Tattersall and Millam suggest that there is a need for breeding Camelina sativa via genetic transformation, they were not able to produce and regenerate transgenic Camelina sativa plants. Therefore, there is a need for a system to transform Camelina plants and subsequently regenerate the transgenic cells into transgenic plants.

Brassica species have been used as common model plants in plant breeding and molecular biology, but because they are prone to pests like Meligethes aeneus, an alternative related plant would be useful. Camelina sativa would provide such a new model plant, which is not sensitive to the pest. Furthermore, Camelina sativa has a relatively small genome, including only 20 chromosomes, which simplifies its use in genetic studies. Classically for example tobacco and Arabidopsis have been used as model plants. However, when compared to Arabidopsis, Camelina sativa provides more plant material following transformation or other manipulations for further experiments. Accordingly, there is a need for a method to transform and regenerate the transformed Camelina sativa cells.

In addition, there is an impeding need to introduce commercial crops to provide vegetable oils for biofuel production without displacing food crops from rich soils. Because Camelina sativa is well suited to marginal soils, this plant species offers an alternative crop that can be grown and harvested in large quantities. However, because of limited breeding success, improvements in Camelina sativa, such as herbicide resistance, increased protein quality, increased oil content, and enhanced agronomic characteristics are lacking. In addition, because Camelina sativa has extremely limited pollen travel and is not a commercial food crop, the ability to transform and produce transgenic Camelina sativa plants is crucial for its further development as a commercial crop.

This invention solves the problems of the prior art. We have developed a method to efficiently transform Camelina sativa explants and regenerate the transgenic plants. Moreover, our invention provides a method that can be used without selection markers, thereby providing selection marker free transgenic Camelina sativa plants.

### SUMMARY OF THE INVENTION

Accordingly, present invention provides a genetic transformation system for Camelina sativa, which would address rapid improvement of this crop for different end-uses, including production of homologous and heterologous recombinant DNA products. Examples of homologous recombinant products comprise unique protein or oil products specific for Camelina sativa, whereas heterologous products include foreign proteins, enzymes, etc.

Present invention also provides a method to produce transgenic Camelina sativa plants without a selection marker. Accordingly, present invention also provides transgenic Camelina sativa plants that do not carry a selection marker gene, such as antibiotic resistance or herbicide resistance genes. This novel method is highly valuable, because it allows insertion in plant genome only target genes and minimizing extra sequences to some nucleotides left from T-DNA borders.

Therefore the present invention also provides a transformation method that does not introduce bacterial or virus sequences of selectable markers into the plant genome. Accordingly the present invention provides transgenic Camelina sativa plants free form bacterial and viral sequences originating from selectable markers.

Yet another embodiment of the present invention is to provide a novel model plant for replacing e.g. Arabidopsis and tobacco. Camelina sativa has a relatively small genome, including only 20 chromosomes, which greatly simplifies its use in genetic studies. Moreover, Camelina transformation and regeneration process according to the method of this invention is fast and reliable.

A further embodiment of the present invention is to provide transgenic Camelina sativa plants, plant tissue, plant cells and cell lines and seed.

The specific advantage of the present method is that it provides efficient genetic transformation of Camelina sativa, reliable and fast regeneration of transgenic plants, and subsequent production of heterologous and homologous gene products. Camelina sativa germinates and grows rapidly and explants can be excised from plantlets after only 10 days from germination. Genetically transformed Camelina sativa plants can be transferred to greenhouse after four weeks from transformation event. The transformation efficiency of Camelina sativa according to the current method is high. The rapid growth of Camelina sativa enables that the transformation method can be scaled up for future applications.

The present invention provides a method to produce transgenic Camelina sativa plants, preferably free from selection markers, and expressing products encoded by the chosen gene(s) of interest. Non limiting examples of such genes of interest are genes that modify the oil profile of Camelina seeds, genes that modify the protein content or quality of Camelina seeds. Yet another example of genes of interest is genes that encode pharmaceutically important molecules.

The present invention provides a novel method to genetically transform Camelina sativa by Agrobacterium-mediated transformation and a subsequent regeneration of transgenic plants. The method and the products and means used in this method are as defined in the claims of the present disclosure and they provide an efficient, reliable and convenient transformation system for producing Camelina sativa crop with improved properties via transgenic improvement and recombinant DNA technologies.

### A SHORT DESCRIPTION OF THE DRAWINGS

FIG. 1 A. shows in vitro cultured Camelina sativa plant. B. Camelina sativa hypocotyls segments transferred into a Petri dish for improved development of first true leaves for explant production.

FIG. 2 shows regenerated shoots of Camelina sativa on leaf segment explants.

FIGS. 3a and 3b depicts GUS expression in callus tissue of Camelina sativa. The arrowheads point to GUS stained inclusions.

FIG. 4. Shows the RT-PCR assay of RNA expression and DNA insertion in GUS positive selection marker free Camelina sativa plants. A. Total RNA samples; B. RT-PCR from 16 samples of total RNA; C. PCR form the same total RNA samples without reverse transcription. PCR product originating from DNA has a size of 466 bp. Product from spliced mRNA has a size of 276 bp.

FIG. 5. shows Camelina sativa plantlets grown in greenhouse conditions. The plantlets are obtained from transgenic shoots recovered and rooted after in vitro selection of transformed explants of Camelina sativa.

FIG. 6. Transgenic Camelina sativa shoots on root elongation medium.

FIG. 7. GUS positive transgenic Camelina sativa shoots that are free from selection marker.

FIG. 8. Depicts transformation vectors A. pCambia 1301 and B. new selection marker free transformation vector pCambia 0301.

### DETAILED DESCRIPTION OF THE INVENTION

Transformation and regeneration methods for Brassica species have been previously disclosed in U.S. Pat. No. 5,463,174. However, even if Camelina sativa belongs to the family Brassica ceae, none of the disclosed methods allow either efficient transformation, or successful regeneration of transgenic Camelina sativa plants. Tattersal and Millam (1999) have developed a method to regenerate non transgenic, wild type Camelina sativa plants, but surprisingly this method cannot be used to transform and regenerate transgenic Camelina sativa plants. Therefore, there is no functional protocol for transforming Camelina sativa and regenerating transgenic Camelina sativa plants.

We have developed an efficient transformation method for plant explants, preferably leaf segments, of Camelina sativa plants grown in vitro by using Agrobacterium-mediated transformation. The method also provides efficient regeneration of transgenic Camelina sativa plants. Moreover, the invention provides an efficient Agrobacterium-mediated transformation and regeneration method for production of transgenic Camelina sativa plants without use of selection markers. Accordingly, the invention provides transgenic Camelina sativa plants that do not contain selection marker genes.

The key elements of the method according to this invention include a number of steps in obtaining the initiation material (explants). These steps include use of seeds collected from controlled conditions, for example from greenhouse or growth chamber grown Camelina plants, sterilization of the seeds, and in vitro cultivation of the plants from which the explants are later obtained. Selection of an Agrobacterium tumefaciens strain and transformation vectors that provide efficient transformation in Camelina sativa tissue is another essential step. The challenging and non obvious step with Camelina sativa transformation was to develop a method to regenerate the transformed tissue to transgenic Camelina sativa plants. Surprisingly we found that the best result is obtained with high sugar concentrations in regeneration medium. The method also includes use of separate root elongation medium.

### Plant Material

According to this invention the starting material is Camelina sativa seeds collected from green house or growth chamber grown Camelina sativa plants. Using only seeds produced in greenhouse or growth chamber is important, because seeds collected from field grown plants surprisingly were not successful material for Agrobacterium transformation. This is probably because field grown seeds may have been contaminated with bacteria, which later prevented successful transformation with Agrobacterium.

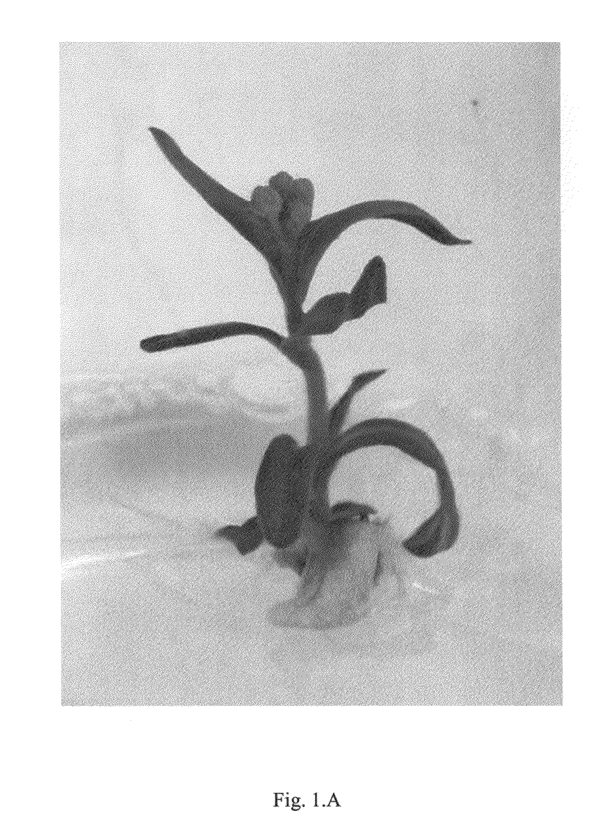

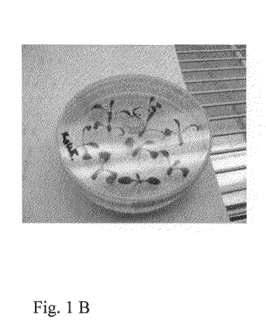

Camelina sativa seeds have a 0.5-1 mm thick hygroscopic polysaccharide surface around the seed that protects the seed for example against fungal and bacterial spores. Camelina sativa seeds therefore require more effective surface sterilization than many other species. Therefore, the seeds were first sterilized 1 min in 70% ethanol and 5-10 min in Na-hypochlorite (2.5% active Cl⁻) with addition of Tween-20, and washed three times in sterile water. Subsequently the sterilized seeds were germinated and grown in sterile jars on Murashige and Skoog (MS) agar medium or an equivalent plant growth medium. Preferably, the seedlings were cut in middle of the hypocotyls and moved to agar plates to grow the first true leaves (FIG. 1B). A segment of the first true leaves were used for explants. Preferably leaves of 10 day old seedlings were used.

### Agrobacterium Vectors

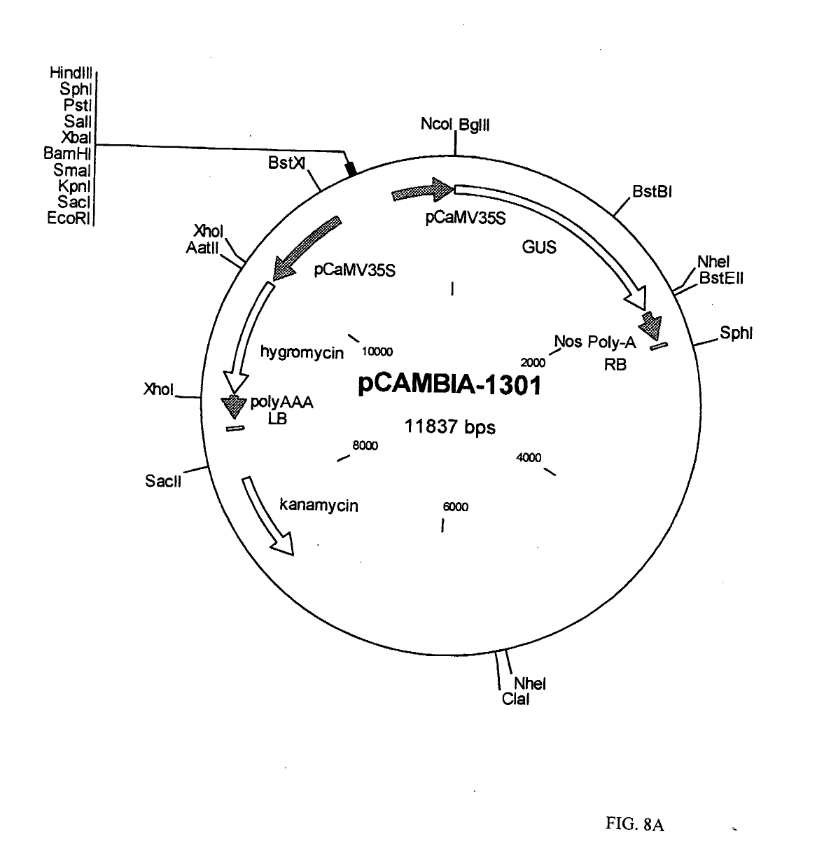

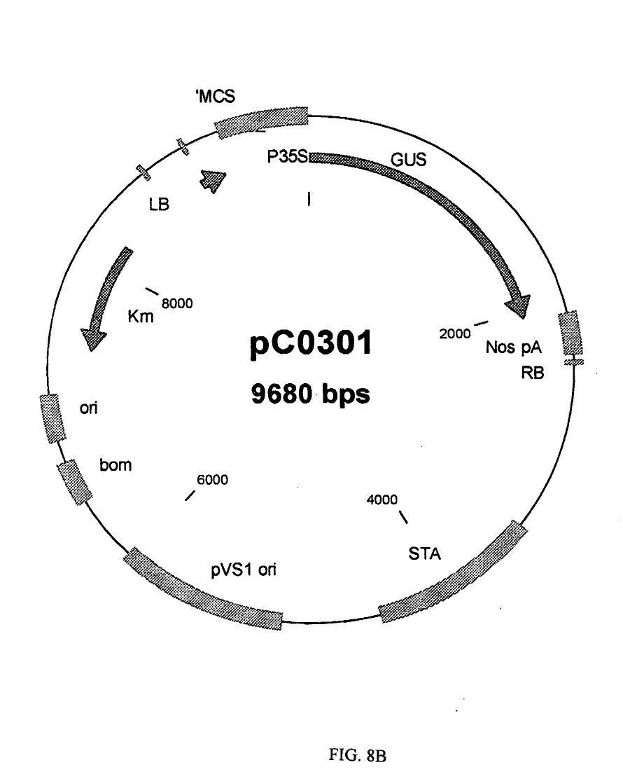

Agrobacterium tumefaciens strain C58C1 containing the plasmid pGV3850 (Zambryski et al., EMBO J. 2:2143-2150, 1983), strain EHA105 (Hood et al., Transgenic Res. 2:208-218, 1993) with the plasmid pTiBo542 and strain LBA4404 with pAL4404 (Hoekema et al., Nature 303:179-180, 1983) were tested for transformation of Camelina sativa. Alternatively, C58 strain containing helper plasmid pGV3850 and binary pC0301vector as described in Example 6 and shown in FIG. 8B was used for transformation. The uidA-gene (β-glucuronidase, GUS) containing an intron (uidA-int) (Vancanneyt et al., Mol. Gen. Genet. 220:245-250, 1990) was cloned into the strains. The uidA-intron-containing gene was used to prevent bacterial GUS expression and it also enabled the testing of GUS-activity at an early stage of transformation. In addition to allow us to visually test the transformation rates, uidA represented here an example of a gene of interest. The co-integrative pHTT294 vector, essentially similar to pHTT370 (Elomaa et al., Bio/Technology 11:508-511, 1993) carrying the uidA-intron-containing gene under the CaMV is 35S promoter (Datla et al., Plant Sci. 94:139-149, 1993), was transferred to an Agrobacterium strain C58C1. Binary pGPTV-HPT and pGPTV-KAN vectors (Becker et al., Plant. Mol. Biol. 20:1195-1197, 1992) with the uidA gene exchanged for the uidA-intron-containing gene under the control of the 35S promoter of CaMV were transformed into Agrobacterium tumefaciens strains EHA105 and LBA4404.

Agrobacterium tumefaciens was grown overnight in liquid YEB (Lichtenstein and Draper, Genetic Engineering of Plants. In: Glover DM (ed.) DNA cloning—a practical approach, vol. 2. Oxford IRL, Oxford, pp 67-119, 1985) medium with shaking supplemented with appropriate antibiotics for each strain. An aliquot (1/100 v/v) of the overnight culture was then inoculated in fresh YEB medium with appropriate antibiotics and bacteria were grown overnight with shaking. An Agrobacterium tumefaciens culture of OD₆₀₀=1.0 was used for transformation.

### Culture Medium

Composition of Murashige and Skoog (MS) plant growth medium:

| Salts: | | Vitamins: | mg/l |
|---|---|---|---|
| | g/l | | |
| NH₄N0₃ | 1.65 | Thiamine | 0.1 |
| KNO₃ | 1.9 | Pyridoxine | 0.1 |
| MgS0₄×7H₂O | 0.37 | Nicotinic acid | 0.5 |
| KH₂P0₄ | 0.17 | Myo-inositol | 100 |
| CaCl₂×2H₂0 | 0.44 | Glycine | 2.0 |
| | mg/l | | |
| H₃B0₃ | 6.2 | Sucrose | 2.0 |
| MnSO₄×4H₂0 | 22.3 | Agar | 7.0 |
| ZnSO₄×7H₂0 | 8.6 | pH 5.6 | |
| KJ | 0.83 | | |
| Na₂MoO₄×2H₂O | 0.25 | | |
| CuSO₄×5H₂O | 0.025 | | |
| CoCl₂×2H₂0 | 0.025 | | |

Plant transformation. Leaf segments of in vitro grown Camelina sativa plants (FIG. 1A) were cultivated for 24 hours on MS-medium or an equivalent medium supplemented with 0.7% agar. All MS culture media were supplemented with 2% sucrose unless otherwise stated, and all in vitro cultures were kept at temperatures of 25° C. (day) and 18° C. (night) under 16 h photoperiod. Subsequently, the explants were immersed for 1-3 min in Murashige and Skoog (MS) solution or an equivalent which had been inoculated with a dilution (e.g. 1/10 vol/vol) of an overnight culture of Agrobacterium tumefaciens. Thereafter, redundant liquid present on the surface of leaf segments was removed using filter paper and the explants were placed on the MS-agar medium supplemented with auxin and cytokinin hormones, 6-benzylaminopurine (BAP) and naphthaleneacetic acid (NAA), for co-cultivation with bacteria for 2 days. After co-cultivation, the explants were washed with water containing cefotaxime (Claforan) (700 mg/l), carbenicillin (200 mg/l) or ticarcillin/clavulanic acid (Duchefa) (100 mg/l). The surfaces of the explants were dried on filter paper and placed on the MS-medium or an equivalent medium for selection and shoot regeneration.

Selection and regeneration. Eventually, cultivation of the explants for two weeks on MS-medium or an equivalent medium supplemented with 0.5-1.5 mg/l 6-benzylaminopurine (BAP) and 0.1-1.0 mg/l naphthaleneacetic acid (NAA) was found to be best for callus, shoot and root formation. Sucrose concentrations of 2-6% gave best results. Thereafter, the whole explants or cut shoots were transferred to Petri dishes containing hormone-free or NAA supplemented MS-medium with 1-4% sucrose concentration, where recovered shoots elongated and started to root.

Recovered transgenic shoots were grown on MS medium or an equivalent medium without hormones or optionally supplemented with 0.1-0.3 mg/l α-naphthaleneacetic acid (NAA) for stimulation of rooting, stem elongation and micropropagation. Sucrose concentration was preferably 1-4%. The exact hormone concentrations varied for different cultivars tested. Selection using hygromycin or alternatively kanamycin was applied preferably immediately after co-cultivation of the explants with Agrobacterium tumefaciens. Antibiotics were used in concentrations ranging between 15-25 mg/l. Optional selection with an antibiotic was carried out for 4-10 days after co-cultivation. It could be seen already after 10-14 days that the leaf segments produced callus and transgenic shoots.

### Results of the Preliminary Experiments in Developing the Transformation Method

Source plants. Field-grown Camelina sativa plants produce seed heavily contaminated and were practically improper for use in the transformation, because leaf explants contained bacteria which prevented successful transformation by Agrobacterium tumefaciens. To achieve good starting material, Camelina sativa plants were grown in greenhouse or growth chamber conditions and seeds were collected from these plants. These seeds were free of contaminations after surface sterilization. Camelina sativa seeds have a hygroscopic polysaccharide surface, which forms a 0.5-1 mm barrier around the seed to protect the seed against fungal and bacterial spores. This particular characteristic of Camelina sativa seed surface requires more effective surface sterilization of seeds compared to many other species. Camelina sativa seeds were immersed in 70% ethanol for 1 min and treated with Na-hypochlorite solution with an addition of Tween-20 (1 drop per 100 ml).

After sterilization the seeds were washed three times in sterilized water and placed on MS agar medium or an equivalent medium without sugars for germination. Germination was assessed 3 days after sterilization. 5-10 min treatment with 2.5% Na-hypochlorite was found best for Camelina sativa seed sterilization.

Sterilized seeds were germinated and grown for 2-3 weeks or preferably 10 days on MS agar medium or an equivalent medium without sucrose and hormones in sterile jars (FIG. 1A). The green leaves served as a source for explants for the transformation. Alternatively, the hypocotyls were cut and placed on Petri dishes on MS agar without sucrose and hormones and the first true leaves were used for explants (FIG. 1B).

Plant transformation. Three different Agrobacterium tumefaciens strains, namely C58C1, EHA105 and LBA4404 were tested. C58ClpGV3850 harbors the cointegrative vector pHTT294.

The strains EHA105 and LBA4404 carried the binary vector pGPTV-HPT. Alternatively C58 strain with helper plasmid pGV3850 and binary pC0301 vector was used. UidA-intron-containing reporter gene was cloned from pGUS-int into all the binary and cointegrative vectors used in the transformation experiments. The uida-int gene was placed under CaMV 35S promoter.

Hypocotyl, cotyledon, leaf and stem segments were tested for affinity to Agrobacterium tumefaciens. Leaf segments had the best transformation capacity and were used in further transformation experiments. Leaves of in vitro grown Camelina sativa plants are rather small in size: 2 to 4 cm long and 0.5-1 cm wide. Therefore, narrow segments of 0.5-1.5 cm were cut across the leaf.

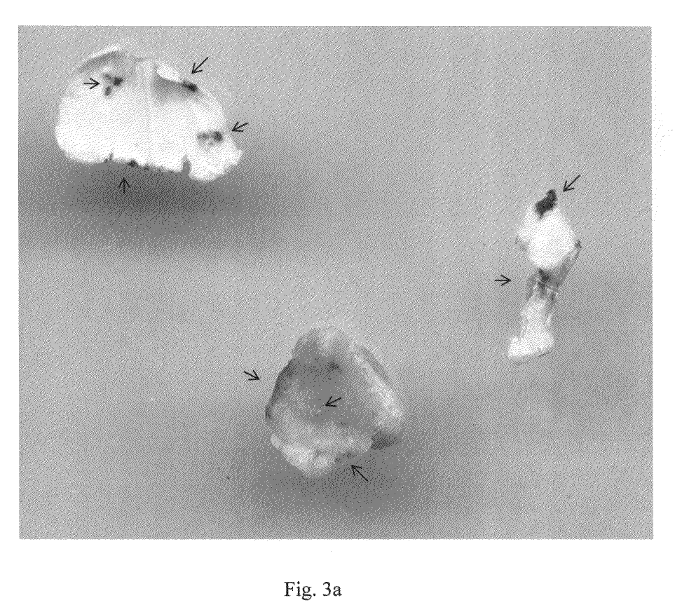

Transformation efficiencies of different Agrobacterium tumefaciens strains were measured as a proportion of blue inclusions in callus one week after inoculation of leaf segments (FIG. 3a).

TABLE 1
Transformation efficiencies of different Agrobacterium tumefaciens
strains. First column: GUS positives/all explants, Second column:
intensive transformation.

| | Blue inclusions | Transformation % |
|---|---|---|
| Agrobacterium | all explants | (intensive) |
| LBA4404pGPTV-HPT | 35/50 | 70% |
| EHA105pGPTV-HPT | 24/50 | 48% |
| C58C1pGV3850 pHTT294 | 33/50 | 66% |

The results of the three transformation experiments, summarized in Table 1, showed that LBA4404 and C58C1pGV3850 strains were effective in transforming Camelina sativa. EHA105 was slightly less effective. The explants infected with LBA4404 or C58C1 strains had large intensively stained blue inclusions. Thus, the strains LBA4404 and C58C1 were used in subsequent transformation experiments.

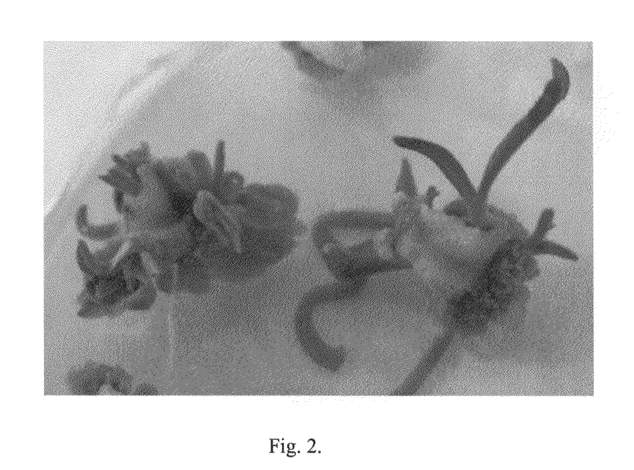

Shoot regeneration. Effects of different hormones on various explants of Camelina sativa (hypocotyl, cotyledon, leaf and stem segments) were tested in preliminary experiments to achieve sufficient shoot regeneration. 6-benzylaminopurine (BAP) and α-naphthaleneacetic acid (NAA) were more effective to induce shoot and root regeneration than kinetin and indole-3-acetic acid (IAA). The regeneration capacity of cotyledons was 30-50% whereas shoots from hypocotyl and stem segments did not regenerate. The best regeneration (100%) was achieved with leaf segments (FIG. 2). The 2,4-dichlorophenoxyacetic acid (2,4-D), gibberellins as well as silver nitrate treatments did not have an effect on shoot regeneration. The best regeneration was achieved with a certain ratio of auxin and cytokinin hormones. For example, the best shoot regeneration of leaf segments of Camelina sativa variety cv. Calena was achieved with the hormone combination of 1.5 mg/l 6-benzylaminopurine (BAP) and 1 mg/ml NAA for 10-14 days and then 1.5 mg/l BAP, while the optimal combination for Camelina sativa variety cv. Calinca was 0.7 mg/l 6-benzylaminopurine (BAP) and 0.3 mg/l α-naphthaleneacetic acid (NAA).

Recovered shoots had a tendency for inflorescence formation and had problems with rooting. To overcome these problems, recovered shoots were cultivated subsequently on MS-medium or an equivalent medium optionally supplemented with auxins (e.g. indole-3-acetic acid (IAA) 1 mg/l). Alternatively, shoots and roots were regenerated simultaneously with the hormone combination of 0.5-1 mg/l 6-benzylaminopurine (BAP) and 0.2-0.7 mg/l α-naphthaleneacetic acid (NAA).

Several different factors were tested for impact on shoot regeneration efficiency. Optimal parameters were found for pH (5.6-5.8), for sucrose content (2-4%), and solidifiers (0.7% agar). Modifications in the concentration of NH4, NO³⁻, K⁺ and Ca²⁺ ions in the standard Murashige and Skoog (MS) medium had no effect nor did the addition of glucose. Culturing the explants on the B5 medium had also no effect on shoot regeneration.

Selection. To prevent Agrobacterium tumefaciens growth on the medium, cefotaxime (Claforan) (500 mg/l), carbenicillin (200 mg/l), ticarcillin/clavulonic acid (Duchefa) (100 mg/ml) or vancomycin (200 mg/ml) were used.

In experiments with selection markers (eg. hpt and nptII genes in transformation constructs and hygromycin or kanamycin respectively in culture medium) it was found that the application of a selection pressure (15-20 mg/l, preferably 10-20 mg/l of antibiotic) preferably for 4-10 days after washing of the Agrobacterium tumefaciens from explants was optimal. First regenerative primordia form on the calli 10 days after cutting of the leaf segments, and selection of transformed tissues should be performed before that. It was found in preliminary experiments that 5-15 mg/l antibiotic prevented morphogenesis of explants. Selection of transformed tissue using 5-10 mg/l hygromycin or kanamycin was not enough. On the other hand, the concentrations of the antibiotic higher than 20-30 mg/l killed the explants too fast for any shoots to recover.

### Analysis of Transformation

The histological GUS assay was performed as described in Example 4 below. The assay enabled the testing of GUS activity almost immediately after co-cultivation with Agrobacterium tumefaciens. Usually, GUS assay was made 4-7 days after co-cultivation with Agrobacterium tumefaciens during the optimization of transformation (FIG. 3). The assay was also performed on regenerated primordia and shoots as well as leaf segments of recovered plants.

PCR analysis was performed as described in Example 4 below. No PCR product was obtained when non-transgenic Camelina sativa DNA was used as template, whereas when using transgenic Camelina sativa an amplification product of 700 nucleotides corresponding to the positive control was obtained which confirmed the presence of transgene in transgenic Camelina sativa plants. RT-PCR was performed as described below in Example 8.

Southern analysis was performed as described in Example 4 below. Presence of the transgene insertion was proved in comparison to DNA of non-transgenic Camelina sativa plant DNA as negative control, and to plasmid DNA carrying the gene sequence mixed with non-transgenic plant DNA as positive control.

In the illustrative examples below, we used uidA reporter gene, which enabled verification of transformation even when a selection marker was not used. However, when selection markers are not used, and a reporter gene is not inserted into the genome, the first screening of regenerated shoots can be performed by using PCR technologies, or immunoassays.

The invention is now described with examples that are not meant to be limiting to the scope of the invention.

### EXAMPLE 1

### Transformation Protocol for Camelina sativa cv. Calena with Agrobacterium tumefaciens Strain LBA4404 Harboring the Binary Plasmid pGPTV-HPT with uidA Intron Containing Gene

The seeds of Camelina sativa plant grown in greenhouse were sterilized by immersing in 70% ethanol for 1 min and then treating for 10 min with Na-hypochlorite solution (3% active Cl⁻) with an addition of Tween-20 (1 drop per 100 ml). After sterilization the seeds were washed three times in sterile water and placed on solid MS-agar medium without sugars for germination. Sterilized seeds were germinated and grown 2-3 weeks on solid MS medium without hormones (FIG. 1). Green leaves served as a source of explants for transformation procedure.

Agrobacterium tumefaciens strain LBA4404 carrying pGPTV-HPT-GUSint vector was grown overnight at 28° C. with shaking in liquid YEB medium supplemented with 50 mg/l kanamycin and rifampicin. Subsequently an aliquot of the culture (1/100 v/v) was inoculated in fresh YEB medium supplemented with 50 mg/l kanamycin and rifampicin and the bacteria were grown overnight with shaking. Agrobacterium culture of OD₆₀₀=1.0 was used in the transformation experiments.

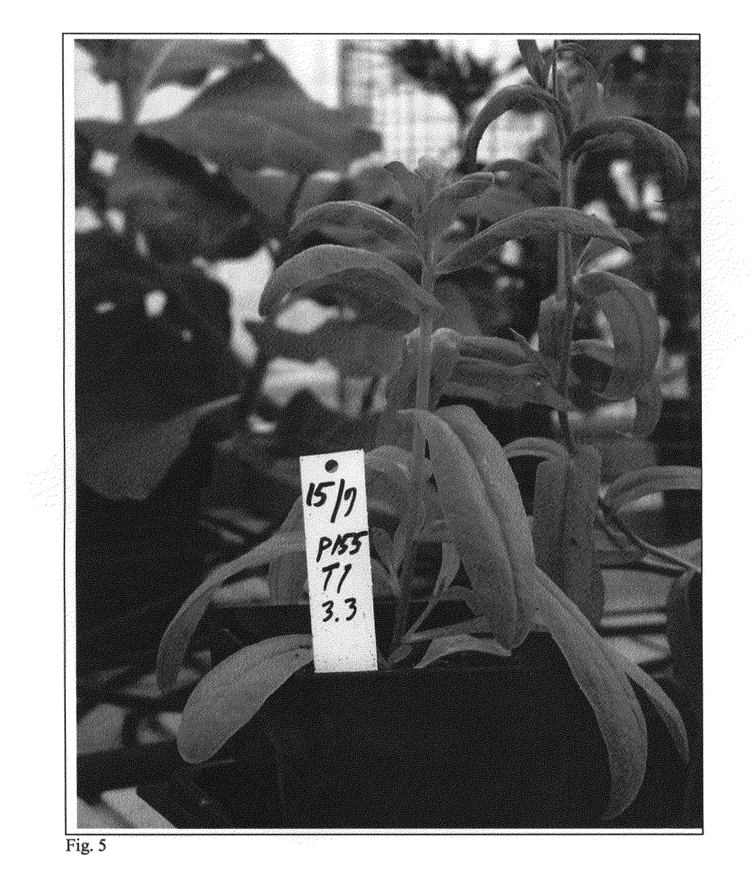

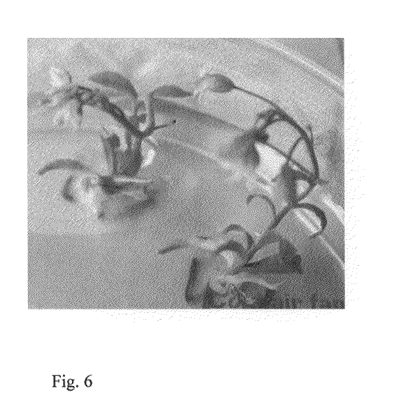

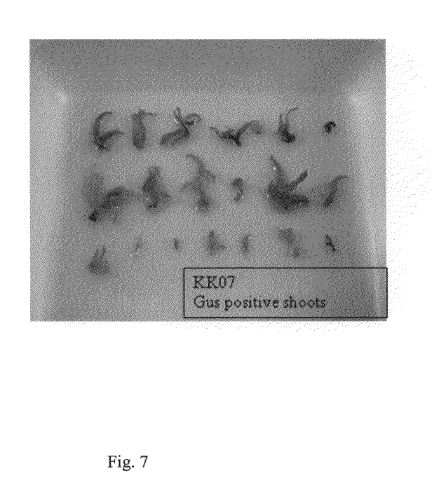

The middle parts of narrow leaves of in vitro grown Camelina sativa plants were used as explants, whereas large leaves were additionally cut in half along the central vein. The leaf segments were cultivated for 24 hours on MS 0.7% agar medium supplemented with 1 mg/l 6-benzylaminopurine (BAP) and 0.2 mg/l α-naphthaleneacetic acid (NAA). All MS-culture media were supplemented with 2% sucrose if not otherwise stated and all in vitro cultures were kept at temperatures of 25° C. (day) and 18° C. (night) under the photoperiod of 16 h. The explants were immersed for 1-3 min in MS-solution inoculated with a dilution (e.g. 1/10 v/v) of the overnight culture of Agrobacterium tumefaciens LBA4404. Redundant liquid on the stem segments was removed with filter paper and the explants were placed on MS-agar medium supplemented with auxin and cytokinin for co-cultivation with bacteria for 2 days. The explants were washed with water containing claforan [cefotaxime) (700 mg/l)] or carbenicillin (700 mg/ml). After two days of co-cultivation, the surfaces of the explants were dried with filter paper and the explants were placed on MS-medium supplemented with hormones [0.7 mg/l 6-benzylaminopurine (BAP), 0.25 mg/l α-naphthaleneacetic acid (NAA)] and 200 mg/l carbenicillin or claforan and 15 mg/ml hygromycin. Two to three weeks old shoots (FIG. 2) were then placed on the normal or half strength MS medium solidified with 0.7% agar and supplemented with 200 mg/l carbenicillin or cefotaxime and optionally with 15 mg/l hygromycin and auxin [indole-3-acetic acid (IAA) 0.5-1 mg/l]. Shoots were transferred to soil and transgenic plants were grown in greenhouse conditions (FIG. 5).

Transgenic plants were tested for uidA (GUS) gene expression with a histological GUS assay and the presence of the transgene was confirmed with Southern analysis.

### EXAMPLE 2

### Transformation Protocol for Camelina sativa cv. Calinca with Agrobacterium tumefaciens Strain C58C1 pGV3850 Harboring the Binary Ti Vector with Kanamycin Selection

### 10 Days Before Excision of the Explants.

Seeds of greenhouse grown Camelina sativa cv. Calinca plants (not older than 4 months) were sterilized and placed in vitro on MS-agar medium without sucrose and grown at temperatures of 25° C. (day) and 18° C. (night) as described in Example 1.

### 1^{st} Day.

A fresh colony of Agrobacterium tumefaciens strain C58C1pGV3850 carrying binary pGPTV-KAN vector containing uicA-int gene under 35S promoter and selectable marker gene nptII, was inoculated in 3 ml of liquid YEB medium supplemented with 25 mg/l rifampicin (Rif) and 50 mg/l kanamycin (Kan). The bacteria were grown overnight with shaking at 28° C.

2^{nd} Day. Pre-Cultivation.

The first leaves (not cotyledons) of in vitro grown Camelina sativa were cut into segments across the leaf and were placed on pre-cultivation plates containing 0.7% MS agar medium supplemented with 2% sucrose, 0.7 mg/l 6-benzylaminopurine (BAP) and 0.3 mg/l alpha-naphthaleneacetic acid (NAA). All dishes were sealed with porous paper tape (Micropore 3M). A 30 μl aliquot of overnight culture of the Agrobacterium tumefaciens was inoculated in 3 ml of fresh YEB medium supplemented with rifampicin (Rif) and kanamycin (Kan).

3^{rd} Day. Agrobacterium tumefaciens Inoculation.

The explants were immersed in liquid MS-medium supplemented with 2% sucrose and inoculated with a 1/10 (v/v) dilution of the overnight culture of Agrobacterium tumefaciens. After 5 min inoculation redundant liquid on the explants was removed with sterilized filter paper.

Explants were placed on MS-medium supplemented with 2% sucrose for co-cultivation with the Agrobacterium tumefaciens for two days at 28° C. in dim light.

5^{th} Day. Washing and Selection.

Explants were washed with water containing 100 mg/l ticarcillin/clavulanic acid (Duchefa). Ticarcillin (Tc) has less negative effect on shoot and root regeneration than cefotaxime (Claforan) and carbenicillin. Ticarcillin was also more effective growth inhibitor of Agrobacterium tumefaciens than vancomycin. The explants were dried with filter paper and transferred onto selection medium containing 0.7% MS-agar medium supplemented with 2% sucrose, 0.7 mg/l 6-benzylaminopurine (BAP), 0.3 mg/l α-naphthaleneacetic acid (NAA), 15 mg/l kanamycin and 50 mg/l ticarcillin/clavulanic acid (Duchefa). Explants were cultured on the selection medium for 4-5 days.

10^{th} Day. Regeneration.

Explants were transferred onto plates containing 0.7% MS agar medium supplemented with 2% sucrose, 0.7 mg/l 6-benzylaminopurine (BAP), 0.3 mg/l α-naphthaleneacetic acid (NAA), and 50 mg/l ticarcillin/clavulanic acid (Duchefa) for shoot and root regeneration for 10-14 days. Tall (3 cm high) plates were sealed with porous paper tape to increase aeration. Simultaneous regeneration of shoots and roots was preferable for effective recovery of transgenic Camelina sativa plants.

20-24^{th} Day. Shoot and Root Elongation.

Explants that formed 0.5-1 cm long leaves (shoots) and roots were transferred on 0.7% MS-agar medium containing 2% or 3% sucrose and 100 mg/l ticarcillin/clavulanic acid without hormones or optionally supplemented with 1 mg/ml 6-benzylaminopurine (BAP) for 5-7 days.

25-30^{th} Day. Transgenic Plant Growth.

Rooted plants were grown in the jar for 2-3 days before transfer to soil. During this period, the plastic cap was removed from the jar and the jar was covered with filter paper to get the plant to accommodate to dry air conditions. Survival in soil was close to 100%. Recovered shoots formed inflorescence and seedpods. Plant tissues were tested for expression of marker gene (GUS) with GUS assay, PCR and Southern blot.

### EXAMPLE 3

### Transformation Protocol for Camelina sativa cv. Calena with Agrobacterium tumefaciens Strain C58C1 pGV3850 Harboring Cointegrative Ti DNA without Selection of Transgenic Tissues

### 10 Days Before Explants Excision.

Seeds of green house grown Camelina sativa cv. Calena plants (no older than 4 months) were sterilized and placed in vitro on MS-medium without sucrose and grown at temperatures of 25° C. (day) and 18° C. (night) as described in Example 1.

### 1^{st} Day.

A fresh colony of C58C1pGV3850 with interned Ti DNA from pHTT-HPT vector containing GUS gene under 35S promoter and hpt selectable marker was inoculated in 3 ml of liquid YEB supplemented with 25 mg/l rifampicin (Rif) and 100 mg/l spectinomycin (Spe) or streptomycin (Str). The bacteria were grown overnight with shaking at 28° C.

2^{nd} Day. Pre-Cultivation.

The first leaves (not cotyledons) were cut into segments across the leaf and placed onto the pre-cultivation plates containing 0.7% MS-agar medium with 2% sucrose supplemented with 1 mg/l 6-benzylaminopurine (BAP) and 0.5 mg/l alpha-naphthaleneacetic acid (NAA). All plates were sealed with porous paper tape (Micropore 3M).

A 30 μl aliquot of overnight culture of the Agrobacterium tumefaciens was inoculated in 3 ml of fresh YEB medium supplemented with rifampicin (Rif), spectinomycin (Spe) or streptomycin (Str).

3^{rd} Day. Agrobacterium Inoculation.

The plant explants were immersed in liquid MS-medium supplemented with 2% sucrose and inoculated with a 1/10 dilution of the overnight culture of Agrobacterium tumefaciens. Redundant liquid on the explants was removed on sterilized filter paper. The explants were co-cultivated with the Agrobacterium tumefaciens for two days at 28° C. in dim light.

5^{th} Day. Washing and Regeneration.

The explants were washed with water containing 100 mg/l ticarcillin/clavulanic acid (Duchefa). Ticarcillin (Tc) has less negative effect on shoot and root regeneration compared to cefotaxime (Claforan) and carbenicillin. It was also a more effective growth inhibitor of Agrobacterium tumefaciens than vancomycin. The explants were dried on the filter paper. Then the explants were placed onto selection medium plates containing 7% MS-agar medium with 2% sucrose supplemented with 1 mg/l 6-benzylaminopurine (BAP), 0.5 mg/l α-naphthaleneacetic acid (NAA) and 50 mg/l ticarcillin/clavulanic acid (Duchefa) for shoot and root regeneration for 2-3 weeks. Tall (3 cm high) plates were sealed with porous paper tape to increase aeration.

20-24^{th} Day. Shoot and Root Elongation.

Explants that formed 0.5-1 cm long leaves (shoots) and roots were transferred onto 0.7% MS-agar medium containing 2% sucrose supplemented with 100 mg/l ticarcillin/clavulanic acid (Duchefa) without hormones or with 1 mg/ml 6-benzylaminopurine (BAP) for 5-7 days. Plates were not sealed with tape.

Regenerated shoots were tested for GUS expression with histological GUS assay. The strain C58C1pGV3850 was the most effective for transformation of Camelina sativa. 100% of the explants were transformed. The average proportion of tissue in each explant showing GUS expression was more than 30%. This level of transformation efficiency enables transgenic plants to be obtained without antibiotic or other selection. GUS activity was seen in 4 shoots out of 123. It means that average of about 3% of shoots regenerated after transformation were transgenic without use of antibiotic selection. Thus, this method can be used for producing transgenic Camelina sativa plants free from antibiotic resistance genes or other selectable marker genes. Encouraged by this result that shows high transformation rate of the explants, even if the number of transformed shoots was not specifically high, we continued experiments to allow transformation of selection marker free Camelina sativa with a successful regeneration method, which is shown in Examples 6, 7 and 8.

### EXAMPLE 4

### Analysis of Transformation

The histological GUS assay was performed on transformed callus and leaf tissue. To prevent GUS expression in Agrobacteria the uidA gene containing an intron was used in transformation experiments. This enabled the testing of GUS activity even immediately after co-cultivation with Agrobacterium tumefaciens. Usually, GUS assay was made 4-7 days after co-cultivation with Agrobacterium tumefaciens during the optimization of transformation (FIG. 3). The assay was also performed on regenerated primordia and shoots as well as leaf segments of recovered plants.

Transgenic plants which showed steady positive GUS expression and grew well under selection conditions were used for PCR analysis of transgene insertion and Southern blot analysis to confirm the transformation events.

### PCR Analysis.

Total genomic DNA was isolated from leaf tissue of transgenic and non-transgenic Camelina sativa plants using DNeasy Plant Mini Kit according to the supplier's instructions (Qiagen). The presence of the uidA and hpt gene in the GUS positive plants was determined by PCR analysis by using 24 nucleotides long primers specific to the coding sequences of uidA and hpt genes. PCR reaction mix contained approximately 1 ng/μl of template DNA and DyNAzyme polymerase (Finnzymes) was used for amplification. PCR program consisted of: 940 for 2 min; 30 cycles of 94° C. for 30 sec, 48° C. for 30 sec and 72° C. for 2 min. Three μl of PCR reaction mixture was run in 0.8% agarose gel containing ethidium bromide at 100 V. No PCR product was obtained when non-transgenic Camelina sativa DNA was used as template, whereas when using transgenic Camelina sativa an amplification product of 700 nucleotides corresponding to the positive control was obtained which confirmed the presence of transgene in transgenic Camelina sativa plants.

### Southern Analysis

Total genomic DNA was isolated from leaf tissue of Camelina sativa plants using DNeasy Plant Midi Kit according to the supplier's instructions (Qiagen). Three μg of DNA from GUS positive Camelina sativa plants was digested with EcoRI and BamHI restriction enzymes. These enzymes cut out a 2 kb uidA gene fragment from the T-region of pGPTV-KAN (-HPT) inserted in the plant genome. Digested DNA samples were separated in a 0.7% agarose (Promega) gel overnight at 15 mA current and transferred to positively charged nylon membrane (Boehringer Mannheim) using vacuum blotter. RNA probes were synthesized using T3 RNA polymerase on the pBluescript vector carrying uidA or hpt gene sequence and labeled with digoxigenin-11-UTP. The membrane was hybridized and developed according to the supplier's instructions (Boehringer Mannheim, The DIG user's guide for filter hybridization). The membrane was prehybridized at 50° C. for 2 h and hybridized at 50° C. in a “DIG Easy Hyb” hybridization solution (Boehringer Mannheim) overnight with a digoxigenin-UTP labeled RNA probe. The concentration of RNA probe was 100 ng/ml. After hybridization the membrane was washed in SSC buffers, blocked and detected using “Anti-Digoxigenin-AP alkaline phosphatase (Boehringer Mannheim). Chemiluminescent detection was done with CSPD-substrate and the membrane was exposed to X-ray film (Boehringer Mannheim). Presence of the transgene insertion was proved in comparison to DNA of non-transgenic Camelina sativa plant DNA as negative control, and to plasmid DNA carrying the gene sequence mixed with non-transgenic plant DNA as positive control.

### EXAMPLE 5

### Improved Transformation of Camelina sativa Plants with Increasing Sucrose Concentration in the Regeneration Medium

Camelina sativa seeds were collected from green house grown plants. The seeds were sterilized as described above and germinated and grown in vitro on MS-medium without sucrose at temperatures of 25° C. (day) and 18° C. (night). Shoots were cut and transferred on Petri dishes for formation of first true leaf and explants were prepared from the first true leave as described above.

Explants were transformed in co-cultivation for 2 days with Agrobacterium C58pGV3850pGPTV-HPT and then washed and placed on selection on 1×MS 0.7% agar media supplemented with 0.7 mg/l BAP, 0.25 mg/l NAA, 15 mg/l Hyg. And 100 mg/l Tic. The sucrose concentration of the medium was 1.0, 1.5, 2.0, 3.0 or 4.0%.

14 days later the explants were transferred from the selection medium to shoot regeneration medium that contained 1.5×MS 0.7% agar, 1.5 mg/l BAP and 150 mg/l Tic. The regeneration medium had the same sugar concentration as the selection medium, except that explants from 2% sucrose were transferred on medium containing either 2%, 4% or 6% sucrose. After 9 days on regeneration medium, the shoot regeneration frequency was calculated. The results are shown below in the Table.

| Sucrose content | % explants with viable shoots of all explants |
|---|---|
| 1% | 21 |
| 1.5% | 7.5 |
| 2% | 6.7 |
| 3% | 31 |
| 2% > 4% | 29.5 |
| 4% | 30.4 |
| 2 > 6% | 50 |

As is evident from the table above, regeneration of viable shoots was highest in higher sugar concentrations. Regenerated shoots were cut and transferred on rooting medium, said rooting medium containing 1.5 MS agar supplied with 0.3 mg/l NAA and either 1.0, 1.5, 2.0, 3.0, 4.0 or 6.0% sucrose. Alternatively the rooting medium contained 1×MS agar supplemented with 0.7 mg/l BAp+0.25 mg/l NAA+150 mg/l Tic and 0.0, 1.5, 2.0, 3.0, 4.0 or 6.0% sucrose. After 23-26 days, 70 to 100% of the shoots were rooted on media containing 1-4% sucrose.

### EXAMPLE 6

### Selection Marker Free Transformation Vector

We designed a selection marker free transformation vector by removing of hpt-gene from the Cambia 1301 transformation vector (FIG. 8A) The new vector pCambia0301 (pC0301) contains only GUS gene between left and right borders of T-DNA (FIG. 8B). The pC0301 vector was electroporated into Agrobacterium tumefaciens strain C58 with helper Ti plasmid pGV3850. Camelina sativa explants were transformed with selection marker free vector as described below.

### EXAMPLE 7

### Transformation of Camelina sativa Plants without Selection Marker

Camelina sativa seeds were collected from green house grown plants. The seeds were sterilized as described above and germinated in sterile jars on agar. Shoots were cut and transferred onto Petri dishes for formation of first true leave and explants were prepared from the first true leave as described above. Explants were transformed in co-cultivation with the Agrobacterium C58GV3850-C0301 for 2 days and then washed and placed on callus induction medium 1.5×MS 0.7% agar+1.5 mg/l BAP+1.0 mg/l NAA+100 mg/l Tic and either 1.0, 1.5 or 2.0% sucrose.

10 days after washing explants were transferred from callus induction media to shoot regeneration medium. The shoot regeneration medium contained 1.5×MS 0.7% agar supplemented with 1.5 mg/l BAP, 150 mg/l Tic and either 1.0, 1.5, 2.0, 3.0, 4.0 or 6.0% sucrose. Explants from callus medium having 1.0 and 1.5% sucrose, were transferred to shoot regeneration medium with the same sucrose concentration. Explants from callus medium having 2% sucrose concentration were transferred to shoot regeneration medium having 2.0, 3.0, 4.0 or 6.0% sucrose concentration. Ten days later the frequency of shoot regeneration was calculated. The results are shown in the table below.

| | % of explants with viable |
|---|---|
| | shoots of all explants having |
| Sucrose content | shoots |
| 1% | 41 |
| 1.5% | 35 |
| 2% | 59 |
| 2 > 3% | 76 |
| 2 > 4% | 76 |
| 2 > 6% | 71 |

As is evident from the results the best rate for shoot regeneration was received when the shoot regeneration medium contained sucrose concentration of 3% or higher.

At the same time that the shoots were cut from the explants, histological GUS assay was conducted with 180 shoots. 13% of the shoots were GUS positive. In other similar experiments the percentage of transgenic shoots was between 11 and 14% when no selection was used. In experiments where hygromycin selection was used the percentage of transgenic shoots was 25-31%, i.e. only twice the percentage without selection.

### EXAMPLE 8

### RT-PCR Assay of the RNA Expression and DNA Insertion in GUS Positive Plants Transformed without Selection Marker

Eight GUS positive shoots from Example 6. were divided in several shoots to grow and root. Two shoots of each transformation event were tested in RT-PCR for DNA insertion and plant mRNA product.

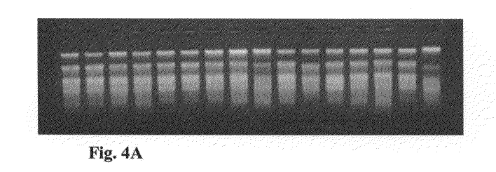

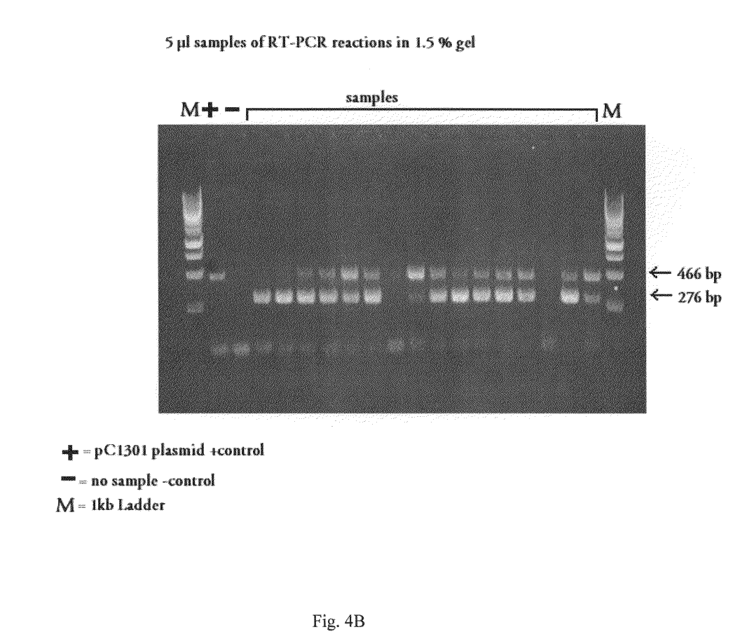

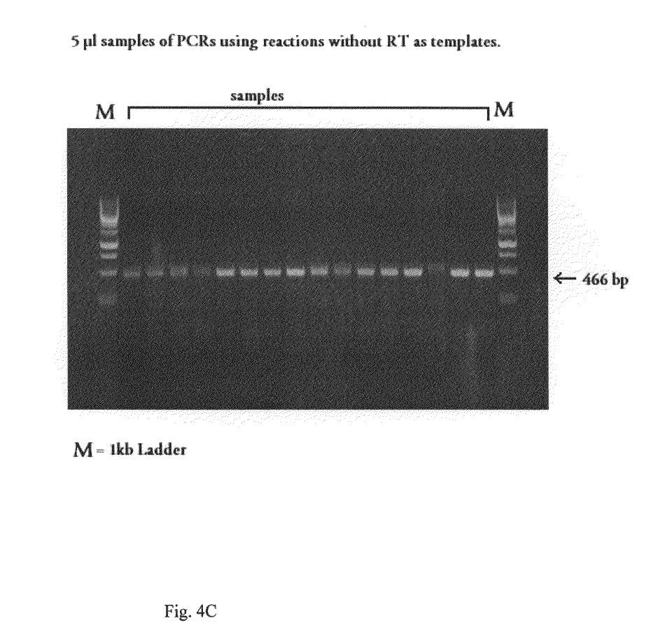

For these purposes plant total RNA was isolated from approximately. 20 mg leaf samples of in vitro shoots using E.Z.N.A Plant RNA kit (Omega Bio-Tek). 250 ng of each sample were denatured in Glyoxal/DMSO RNA loading buffer (Ambion) containing SYBR nucleic acid stain (Molecular Probes) as is shown in FIG. 4A.

1 μg of each RNA sample was reverse transcribed with RevertAid RNaseH-M-MLV reverse transcriptase 200 u (Fermentas) in 25 μl reactions consisting in addition to enzymes, own1× buffer, 1 mM dNTPs, 2 μM random nonamer primers (Sigma-Aldrich), 1.5 μl D(+) trehalose (saturated at room temperature), 800 mM D(+) sorbitol, 10 u SUPERase-in RNase inhibitor (Ambion). Samples were incubated 25° C. 5 min., 37° C. 5 min, 42° C. 5 min., 55° C. 5 min., 93° C. 3 min.

2 μl of each RT-reactions was used as template in 20 μl PCR reactions using Dynazyme II polymerase 1 u (Finnzymes) in it's own 1× buffer 100 μM dNTPs (˜the same amount comes with the template from RT-reactions) 2% DMSO, GUS-5′-F and 250 nM GUS-e2-R primers. Program: 95° C. 4 min., 35×[(95° C., 30 s), (52° C. 20 s), (72° C., 30 s)].

The primers for RT-PCR were designed to flank the intron in the field of coding sequence of GUS gene. In the resulting PCR product from genomic DNA or bacterial contamination will be 466 bp in size, whereas the RT-PCR product from plant mRNA will be 276 bp in size because of processing the intron. In FIG. 4B we clearly see that most of the RNA samples produced the right size (276 bp) amplification product. Positive plasmid DNA control gave the unprocessed size of amplification product (466 bp).

In FIG. 4C we see the PCR amplification products without the Reverse transcription reaction. The production of the larger 466 bp band and absence of 276 bp clearly shows that smaller band is produced from plant mRNA through reverse transcription.

What is claimed is:

1. A method to transform and regenerate Camelina sativa plants, said method comprising the steps of:
a) Providing sterilized Camelina sativa seeds collected from a plants grown in controlled conditions;
b) Germinating the seeds on agar in sterilized conditions and growing in vitro seedlings;
c) Obtaining explants from the in vitro grown seedlings;
d) Inoculating the explants with Agrobacterium tumefaciens strain containing at least one recombinant DNA construct;
e) Cocultivating the explant with the Agrobacterium strain;
f) Transferring the explants to a callus forming medium, said medium being supplemented with hormones and containing 2% sucrose;
g) Transferring the explants to a shoot regeneration medium, said medium being supplemented with hormones and containing 2-6% sucrose;
h) Transferring the shoots to a root elongation medium, said medium being supplemented with hormones and containing 1-4%
i) Transferring the regenerated shoots into soil and growing them to transgenic Camelina sativa plants.

2. A method to obtain selection marker free transgenic Camelina sativa plants, said method comprising the steps of:
a) Providing sterilized Camelina sativa seeds collected from plants grown in controlled conditions;
b) Germinating the seeds on agar in sterilized conditions and growing in vitro seedlings;
c) Obtaining explants from the in vitro grown seedlings;
d) Inoculating the explants with Agrobacterium tumefaciens carrying a plant transformation vector comprising one or more genes of interest and being free from selection marker genes;
e) Cocultivating the explant with the Agrobacterium;
f) Transferring the explants to a callus forming medium, said medium being supplemented with hormones and containing 2% sucrose;
g) Transferring the explants to a shoot regeneration medium, said medium being supplemented with hormones and containing 2-6% sucrose;
h) Transferring the shoots to a root elongation medium, said medium being supplemented with hormones and containing 1-4% sucrose; and
i) Transferring the regenerated shoots into soil and growing them to transgenic Camelina sativa plants.

3. A transgenic Camelina sativa plant, obtained by the method of claim 1.

4. A selection marker free transgenic Camelina sativa plant, obtained by the method of claim 2.

5. A transgenic seed of Camelina sativa plant obtained from the plant of claim 3.

6. A selection marker free transgenic seed of Camelina sativa plant obtained from the plant of claim 4.

7. The seed of claim 6, expressing one or more heterologous or homologous recombinant products encoded by the gene(s) of interest.
